(19) United States
(12) Patent Application Publication
(10) Pub. No.: US 2018/0353279 A1
(43) Pub. Date: Dec. 13, 2018

(54) Exchange wire anchored by a self-expanding retrievable stent and method of use

(71) Applicant: Daniel E. Walzman, Bergenfield, NJ (US)

(72) Inventor: Daniel E. Walzman, Bergenfield, NJ (US)

(21) Appl. No.: 15/530,131

(22) Filed: Dec. 5, 2016

(51) Int. Cl. A61F 2/01; A61F 2/962; A61F 2/90

(52) U.S. Cl. CPC A61F 2/013; A61F 2002/9528; A61F 2/90; A61F 2/962

(57) ABSTRACT

An anchored exchange wire and optional filter assembly and method comprising a wire, with or without a temporary filter, anchored and fixed in position by an attached self-expanding stent. The device is designed to facilitate the safer delivery of endovascular devices over the wire, while minimizing risks that can be associated with unwanted movement of the associated wire and/or filter.

### BACKGROUND OF THE INVENTION

### Field of the Invention

The present disclosure relates to intra-luminal stents without balloon anchors. More particularly the present disclosure relates to self-expandable neurovascular stents employed for endovascular treatment of aneurysms, atheromatous lesions, emboli or stenoses, especially intracranially.

### Background

Stents can expand the interior diameter of vessel. The present invention is adapted to intracranial disease, but is also applicable in certain cardiac and peripheral applications.

Most current techniques to exchange interventional neurovascular devices over a wire use a simple exchange-length wire. Failure to provide an effective anchor may allow the wire to injure the lumen, i.e., the interior wall of a vein or artery by friction or puncture. This may result in a vasospasm, stroke or other adverse effect. It may also result in proximal wire migration, and loss of desired treatment positions and access. An anchor stent may prevent such undesired wire movement during exchanges. An anchor stent may also prevent movement of other medical devices inserted within a vein or artery, which movement may cause the same or worse effects than movement of the wire. Migration of removable filters that capture embolic or similar residues during procedures involving catheterization can have similar adverse effects.

The ability to accurately affix a medical device to a location within an artery is conventionally dependent upon anchoring a delivery system associated with said device. Said anchoring normally occurs at a point which is most distant from the point of origin of said delivery system (the distal location). For example, when a catheter is used as a delivery system, a distal balloon inflation is sometimes the anchoring element.

A stent is a small mesh tube which is most commonly used to treat narrow or weak arteries. Another use is as a scaffold for aneurysm coiling in which an intracranial stent, which is a metal mesh device in the shape of a pipe or tube placed inside the parent artery at the site of an aneurysm to cover the neck of the aneurysm. This helps to keep coils placed in the aneurysm sac to stay inside the sac.

Generally, a stent is a medical device disposed in a lumen of a vessel. The stent is made of metal having super-elastic and springy characteristics. Typically, a stent is inserted into a lumen of a blood vessel using a catheter in a not-expanded state, and is then expanded at a target location, either using a balloon or automatically.

Since an artery actively moves blood through it, a stent positioned in an artery can slip away and migrate from its original installation place as time passes, so that complications arise. In order to solve the above problem, stents have been anchored to the wall of a blood vessel using a plurality of screws or barbs, or outwardly extending flared extensions at their ends.

There are known shortcomings to balloon anchoring. The balloon anchoring technique was initially described by Fujita in 2003 as inflation of a balloon in the side branch of a target coronary vessel to facilitate equipment delivery to a target lesion (see Fujita S, Tamai H, Kyo E, et al. New technique for superior guiding catheter support during advancement of a balloon in coronary angioplasty: The anchor technique. Catheter Cardiovasc Intery 2003; 59:482-488.) Di Mario in 2008 details coaxial and distal variations of the balloon anchoring technique. In coaxial anchoring variation, a balloon is inflated proximally in the target coronary vessel to enhance the penetration capacity of a guidewire. In distal anchoring variation, a balloon is inflated distal to or at the target lesion to enhance support for equipment delivery (see Di Mario C. Techniques to enhance guide catheter support. Catheter Cardiovasc Intery 2008; 72:505-512).

Subsequently, various applications of the distal balloon anchoring technique for enabling deep intubation of a guide catheter, stabilizing the antegrade or retrograde guide wire in chronic total occlusion interventions, enabling stent delivery through challenging anatomy and facilitating delivery of a femoral sheath to the contralateral femoral artery have been documented (see Mahmood, A. Applications of the Distal Anchoring Technique in Coronary and Peripheral Interventions Cath Lab Digest October 2011 Volume 19-Issue 10). Similarly, distal balloon anchoring has been documented for peripheral, carotid, renal and venous interventions, among others.

The use of balloon inflation anchors entails certain inherent short comings which result in dangerous risks and complications for the patient. These include: embolization, or the launching of debris into the bloodstream; Arterial rupture from over-inflation of a balloon catheter or the use of an inappropriately large or stiff balloon, or the presence of a calcified target vessel; Hematoma or pseudoaneurysm formation at the access site; Radiation Injuries Radiation induced injuries (burns) from the X-Rays used and may also provide a less durable treatment for atherosclerosis, and be more prone to restenosis, relative to vascular bypass or coronary artery bypass grafting.

The use of balloon anchoring may also result in prolonged periods of ischemia between inflation of the distal trapping balloon and delivery of the medical device. This is in addition to possible injury at the site of distal balloon inflation.

Despite shortcomings, the prior art has taught way from the use of retrievable stents as anchors.

Prior art teaches that stents must be anchored. For example, US 20080208317 A1 discloses a stent supporting device. Said invention discloses the need to anchor stents. In particular, said invention relates to a stent supporting device, and more particularly to a stent supporting device, capable of securely supporting a stent lest the stent move.

Similarly, the Biodegradable, bioabsorbable stent anchors invention US 20120283811 A1 teaches that metal stents may be used to maintain a pathway within a bodily lumen. However, in many bodily areas, such stents are susceptible to migration from the area in which originally deployed. Such migration is generally undesired because the stent may damage surrounding tissue and may no longer maintain a pathway of the desired lumen.

Also, the Medical Device Fixation Anchor Suited for Balloon Expandable Stents invention US 20100324665 A1 which teaches that various medical devices require some form of fixation or anchoring to a targeted site. Common anchoring means include barbs, hooks, sutures, outwardly extending flared extensions, or other features used to attach a device to the surrounding anatomy. In particular, the invention reports that stents are known in the art to require anchoring accessories.

In short, the prior art teaches that stents need anchors. Thus, said art teach away from the proposition that stents are self anchoring. There is therefore a need for a novel, effective anchor technique for exchanging devices in intraluminal procedures.

### SUMMARY OF THE INVENTION

The current invention discloses a retrievable stent as an anchor. One embodiment of the current invention comprises a retrievable stent that acts to anchor an attached wire in place, so the wire can be used for the delivery and/or exchange of other endovascular medical devices with lower risk of vessel injury, spasm, and/or loss of desired position that can be caused by unwanted movement of the wire.

One embodiment of the current invention includes a filter member to be disposed near or overlapping the anchor stent.

An object of this invention is to provide a secure, retrievable anchor during intraluminal procedures.

Another object of this invention is to avoid adverse or severe adverse effects or death that may caused by irritation to the intraluminal lining, or even vascular rupture, or release of particulate matter by movement of a wire, guide or exchange wire, or migration of a device introduced on such a wire.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a front elevational view of an embodiment of the current invention with a self-expanding anchor stent and filter member disposed upon an elongated wire.

FIG. 2 is an elevational view of the elongated wire with an anchor stent disposed thereon.

FIG. 3 is an elevational view of the anchor stent member (expanded) of the current invention.

FIG. 4 is an elevational view of the filter element (expanded) of the current invention.

FIG. 5 is a cross-sectional view of a sample undeployed embodiment of current invention depicting compressed stent and filter within an introduction sheath.

### DETAILED DESCRIPTION OF THE INVENTION

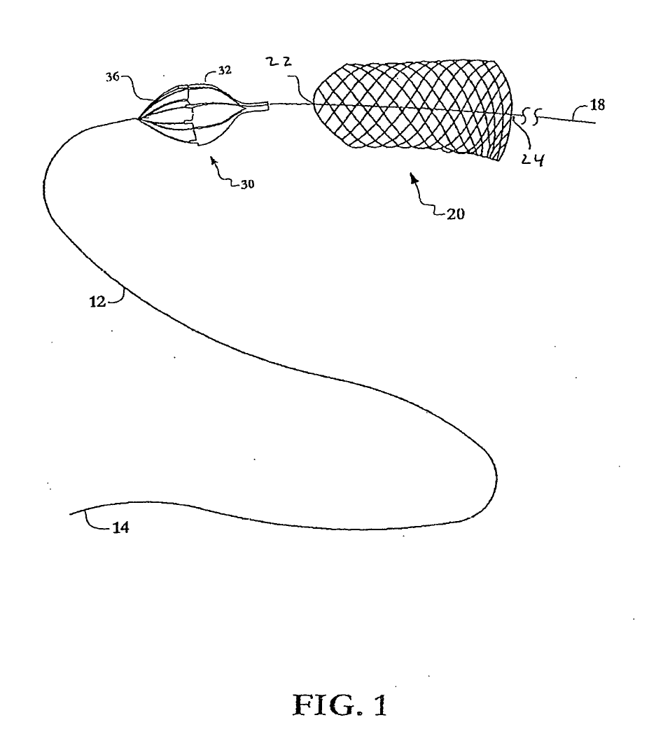

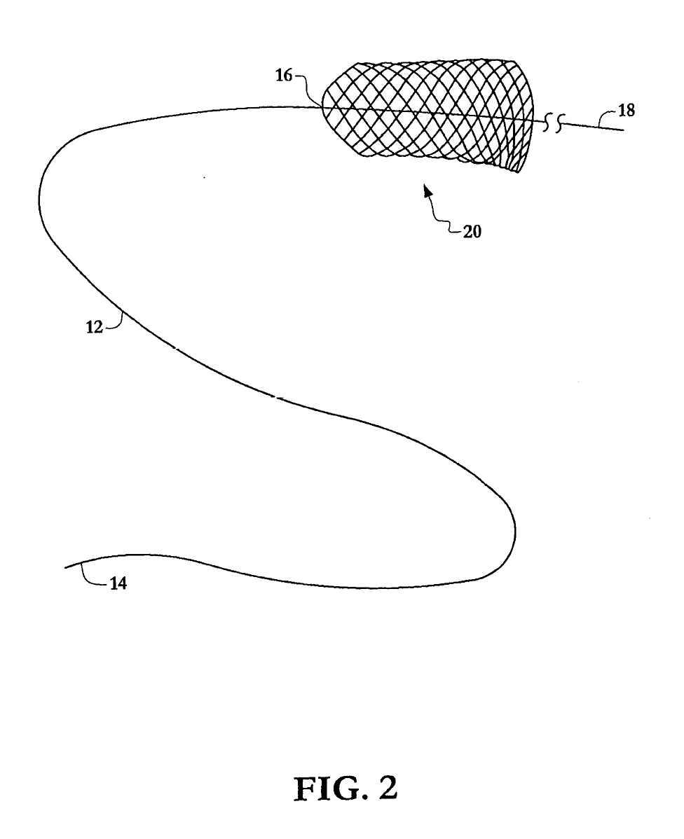

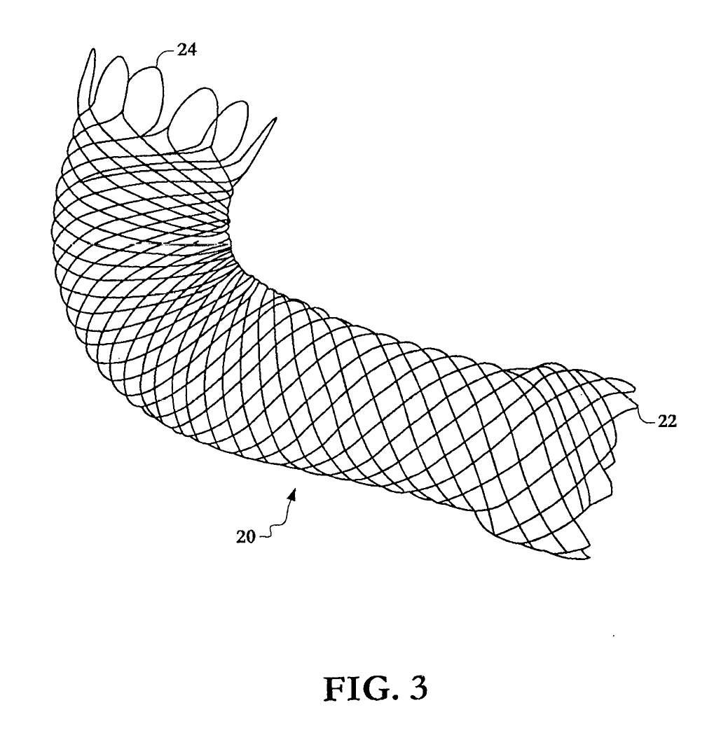

Referring now to FIGS. 1 and 2, the present disclosure depicts an elongated wire 12 that is inserted percutaneously into an artery and passed by the physician through a delivery catheter to the lumen of a target vein or artery to facilitate subsequent delivery of other therapeutic devices. Wire 12 has a proximal 14 and distal end 18. The preferred embodiment includes a self-expanding anchor stent 20 as shown in FIG. 3, having a first end 22 and a second end 24. An optional filter member 30 is shown in FIG. 1 disposed upon wire 12. The proximal first end 22 of self-expanding anchor stent 20 is shown coupled to wire 12. In different embodiments wire 12 may pass through anchoring stent 20 or be coupled therewith at proximal end 22 or elsewhere along the stent. The elongated wire 12 is used to remove and insert devices during the procedure and may be termed an “exchange wire.”

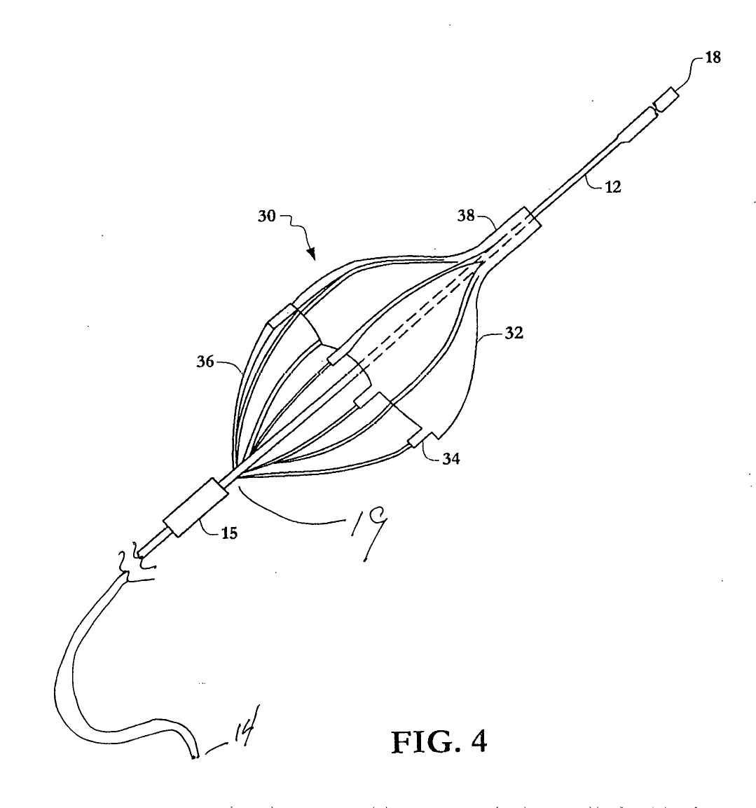

As best shown in FIG. 4, one embodiment of the present invention includes a filter member 30 to capture residues carried in the bloodstream. Filter 30 is disposed upon wire 12 passing therethrough, deployed from holder 15, and disposed upon wire 12 at the proximal end 19 of filter 30. Said filter 30 is shown with a permeable filter capture mesh 32 coupled at connectors 34 to struts 36 that exert radial force upon deployment to expand capture mesh 32 within the lumen.

Typically, filter 30 is deployed with blood flowing (not shown) in the direction from proximal tip 15 of filter 30 to distal end 38 through filter 30 and stent 20, over and around wire 12. The force of blood flow, and the movement of devices over the wire around bends in the vessel anatomy, often cause movement of wire 12 or migration of the filter 30. Even slight movement of filter 30 can irritate the intraluminal walls, release particles, or cause vessel spasm, injury or rupture, with potentially harmful or even fatal effects. Hence, it is important to anchor wire and filter during procedures.

When deployed, stent 20 is expanded to exert radial force against the target arterial or veinal lumen (not shown). Said force anchors stent 20 to minimize movement of wire 12 and, when present, will also prevent migration or movement of filter 30. Anchor stent 20 may be disposed proximally or distally to filter 30, or stent 20 may overlap filter 30, to stabilize filter 30 in the target position within the lumen.

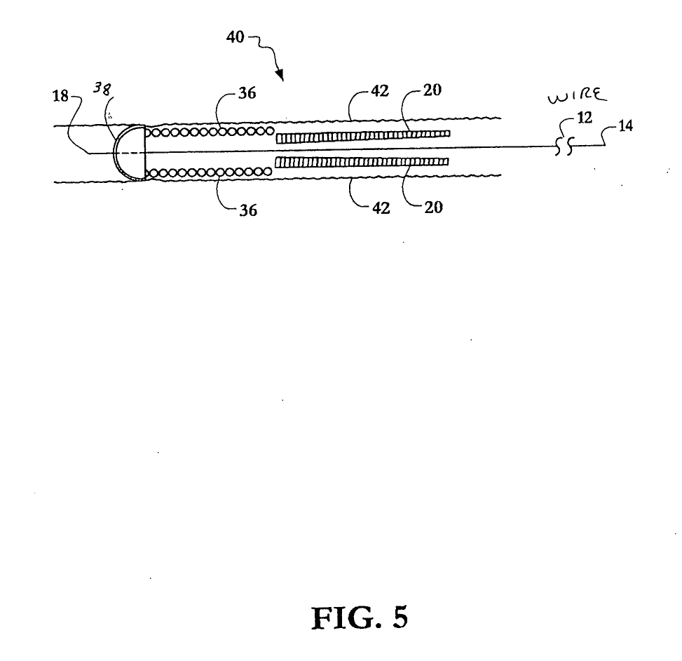

FIG. 5 depicts an undeployed assembly 40 of wire 12 with anchor stent 20 and filter members 36 and 38 disposed thereon in their compressed state. The undeployed assembly 40 is encased within introduction sheath 42. Introduction sheath 42 is typically of a plastic or other material used in catheters through which intraluminal wires and devices are passed.

By way of non-limiting method of using a self-expanding anchor stent with a wire, a larger catheter is first guided into the more proximal artery, then a smaller catheter of choice is delivered, through the larger catheter, to the target location over the wire of choice.

The smaller wire is now removed. Then the anchor device is advanced with the attached exchange-length wire through second the smaller catheter. The anchor stent with attached exchange wire, with or without an attached filter, is unsheathed and left in place, and the smaller catheter is removed.

Other therapeutic devices are now delivered over wire anchored by the self-expanding anchor stent 20. Complete the therapeutic procedure and remove associated delivery tools.

Deliver resheathing catheter over wire, and recapture the self-expanding anchor stent 20. If filter 30 is present, recapture it also. Remove catheter with wire 12 and anchor stent 20 on it. Alternatively, if there is a problem removing said anchor stent 20, the anchor stent may be detached, in some versions, and the wire removed.

In some therapeutic applications, such as several existing undeployed carotid stent assemblies, a custom filter is already preloaded into the delivery catheter (not shown), with a short wire extending from the catheter. A similar assembly can be used with this invention, and then the catheter with preloaded wire, anchor stent, and filter is delivered directly through the guide catheter to the target location. Then the anchor stent 20 and filter 30 is unsheathed as before, removing the catheter while leaving in the wire 12 with the anchor stent 20 and preloaded filter on it. This assembly can then be used to deliver other therapeutic endovascular devices. After this is completed the assembly can be resheathed by advancing a resheathing catheter over it. If there is a problem resheathing said anchor stent 20, the anchor stent may be detached and the wire 12 and preloaded filter alone can be resheathed and removed.

The anchored exchange wire and anchoring self-expanding stent of claim 1, wherein the self-expanding anchor stent or stents are detachable by mechanical, electrolytic, hydrostatic or means known in the art.

The present invention uses a stent with an exchange wire. Said wire has variable stiffness options. Said wire may or may not have at least one small, atraumatic ball at tip of at least one stent strut. Said wire may have a detachable stent which, in the event of difficulties, aids in recapture of wire by allowing detachment of the stent, when desired. The present invention teaches the possibility of various lengths and diameters of said wire, and the option of using a docking wire assembly to elongate the wire as well. A filter may be at the distal end of the wire, or anywhere along the wire, but such filter is optional.

The present invention teaches a wire and anchoring stent designed for intracranial arterial use, wherein the wire has a diameter of 0.006 in.-0.018 in. and the self-expanding attached anchor stent has a fully expanded inner luminal diameter of 0.7 mm-7 mm. Another embodiment of the wire and anchoring stent designed for intracranial arterial use comprises a wire having a diameter of 0.006 in.-0.038 in. and the self-expanding attached anchor stent having a fully expanded inner luminal diameter of 0.7 mm-11 mm. In a still further embodiment, the wire and anchoring stent designed for intravascular cardiac use, the wire has a diameter of 0.006 in.-0.050 in. and the self-expanding attached anchor stent has a fully expanded inner luminal diameter of 0.7 mm-80 mm. In yet another embodiment, the wire and anchoring stent designed for peripheral vascular intravascular use the wire has a diameter of 0.006 in.-0.050 in. and the self-expanding attached anchor stent has a fully expanded diameter of 0.7 mm-120 mm.

The wire and anchoring stent of the designed for intracranial, intravascular over-the-wire exchanges, the wire has a length of 230 cm or longer. An alternative embodiment for intracranial, intravascular “rapid-exchange” exchanges, has a wire with a length of 110 cm or longer.

The stent exchange wire of the preferred embodiment would have an approximate 0.075 to 0.006 inch diameter, with the preferred diameter for intracranial work being wire in the approximate range of 0.006 inches to 0.018 inches in diameter.

The present invention may also have a filter 30. Said filter will include a short self-expanding stent proximal thereto, or an extending along it, and/or positioned distal to it.

The present invention can be used for at least two purposes. One is to minimize movement and migration of a wire during an exchange. In particular, the attached stent acts as an anchor for the wire. A second use of the present invention is to prevent the movement or migration of a temporary filter used during an endovascular procedure. The stent acts as the anchor for the filter. In neither of these cases is any other element needed to anchor these devices, other than the stent. The present invention teaches that the stent is the anchor, unlike the prior art which teaches the need to use an anchor for stents.

It will be understood that the above particular embodiment is shown and described by way of illustration only. The principles and the features of the present disclosure may be employed in various and numerous embodiments thereof without departing from the scope and spirit of the disclosure as claimed. The above-described embodiment illustrated the scope of the disclosure but does not restrict the scope of the disclosure.

1. An assembly comprising: an elongated wire for exchange and delivery of at least one endovascular device; and an attached stent; wherein said wire being anchored by the attached stent.

2. The assembly of claim 1, wherein said wire has a diameter of 0.006 in.-0.018 in. and wherein said stent has a fully expanded inner luminal diameter of 0.7 mm-7 mm.

3. The assembly of claim 1, wherein said wire has a diameter of 0.006 in.-0.038 in. and wherein said stent has a fully expanded inner luminal diameter of 0.7 mm-11 mm.

4. The assembly of claim 1, wherein said wire has a diameter of 0.006 in.-0.050 in. and wherein said stent has a fully expanded inner luminal diameter of 0.7 mm-120 mm.

5. The assembly of claim 2, wherein the wire has a length of at least 230 cm.

6. The assembly of claim 2, wherein the wire has a length of at least 110 cm.

7. The assembly of claim 1, wherein the assembly includes a plurality of self-expanding support stents.

8. The assembly of claim 1, wherein the stent or stents are detachable.

9. The assembly of claim 1 wherein the at least one device comprises at least one filter.

10. The assembly of claim 9 wherein the at least one filter is disposed proximal to said stent.

11. The filter of claim 9 is disposed distal to said stent.

12. The filter of claim 9 overlaps said stent.

13. A non-limiting method of using an anchored exchange wire, comprising the steps of
(a) guiding a catheter into the more proximal artery,
(b) selecting a delivery wire dimensioned for a therapeutic target location,
(c) delivering a preferred catheter to said target location over said delivery wire,
(d) removing said delivery wire,
(e) advancing a self-expanding anchor device with attached exchange-length wire of desired length and diameter,
(f) unsheathing said anchor device and wire,
(g) removing said catheter,
(h) delivering at least one therapeutic device over said anchored, exchange-length wire,
(i) completing therapeutic procedure,
(j) removing delivery tools,
(k) delivering a resheathing catheter over said wire,
(l) recapturing said anchor device with attached wire, and
(m) removing said re-sheathing catheter with said exchange-length wire and anchoring stent.

14. The method of claim 13, adding the further step of detaching said anchor stent and then removing the exchange wire.

15. The method of claim 14, adding the further step of removing said filter.

16. The filter assembly of claim 9 wherein the at least one filter is disposed overlapping said stent.
